(19) United States
(12) Patent Application Publication
(10) Pub. No.: US 2016/0215330 A1
(43) Pub. Date: Jul. 28, 2016

(54) Nucleic-Acid-Sequence Determination Device and Nucleic-Acid-Sequence Determination Method

(71) Applicant: HITACHI HIGH-TECHNOLOGIES CORPORATION, Minato-ku, Tokyo (JP)

(72) Inventors: Tsuyoshi SONEHARA, Tokyo (JP); Tomohiro SHOJI, Tokyo (JP)

(21) Appl. No.: 15/026,150

(22) PCT Filed: Oct. 1, 2014
(86) PCT No.: PCT/JP2014/076251

§ 371 Date: Mar. 30, 2016

(30) Foreign Application Priority Data
Oct. 10, 2013 (JP) 2013-212729

(51) Int. Cl. C12Q 1/68; G01N 21/64

(52) U.S. Cl. CPC C12Q 1/6869; G01N 21/6428; G01N 2201/0636; G01N 2201/062; G01N 2201/06113; G01N 2201/068; G01N 2021/6439

(57) ABSTRACT

A nucleic-acid-sequence determination device equipped with two light sources having different wavelengths, two detectors, and an optical system for irradiating a sample with light from the two light sources and guiding fluorescent light from a nucleic acid in the sample to the two detectors. The optical system is provided with a dichroic mirror for causing the fluorescent light from the nucleic acid in the sample to split, and guiding the split light to the two detectors. The dichroic mirror has a transition wavelength from transmission to reflection in two locations, namely: between light-emitting bands of two types of short-wavelength fluorescent dye and light-emitting bands of two types of long-wavelength fluorescent dye.

### TECHNICAL FIELD

The present invention relates to a nucleic-acid-sequence determination device and a nucleic-acid-sequence determination method of a cluster scheme of causing a chemical reaction of a DNA sample with a reagent on a substrate and analyzing the DNA sample based on images of fluorescence emitted from the DNA sample.

### BACKGROUND ART

A nucleic-acid-sequence determination device (DNA sequencer) of a cluster scheme repeats, on a substrate, complementary DNA extension reaction on amplified sample DNA. The DNA to be extended is modified with four fluorescent bodies with different light-emitting bands such that the sequences thereof can be recognized. A substrate is irradiated with excitation light for each extension reaction, and an image of the fluorescent light emitted from the extended complementary DNA is captured by a camera with an imaging sensor, such as a CCD sensor or a C-MOS sensor, mounted thereon. A type of the fluorescent body is determined based on a color of the image, and a sequence of the sample DNA is determined.

Since the number of types of the fluorescent bodies is four so as to correspond to the types (adenine, guanine, thymine, and cytosine) of bases in DNA, four images of four colors, namely red, yellow, green, and blue are typically captured in a single field of view (referred to as a panel) of the substrate. In addition, red, yellow, green, and blue simply mean four bands with different wavelengths, and do not necessarily correspond to the chromatic sensation of humans. An operation of moving the substrate after imaging one panel and imaging the next panel is repeated. The number of panels to be imaged for one sample is from several hundreds of panels to several thousands or panels. The operation is repeated the number of times corresponding to the base length (several hundreds) in DNA to be analyzed. Therefore, the total number of images to be captured is from several hundreds of thousands to several million for one sample. Information such as a sequence and intensity of colors of emitted fluorescent light is extracted from the captured image data, and a DNA sequence is determined.

According to the most orthodox method of capturing images of the four colors for one panel, four filters with different transmitting regions are mechanically switched, and the images are successively captured by a single imaging sensor as described in PTL 1. The scheme is referred to as a filter wheel scheme. The scheme is also used in a general-purpose fluorescent microscope and is a versatile method. However, since the images are successively captured, and it is not possible to ignore the time required for mechanically switching the filters (exposure time for one image is about 0.1 seconds, and time required for mechanically switching the filters is similar even if the switching is performed as fast as possible), imaging time for one panel is long. As a result, the total imaging time ranges from several days to a week in a case of a nucleic-acid-sequence determination device of the cluster scheme that images a significantly large number of panels, which is one of the most important reasons of an increase in analysis time due to the time required for the imaging. In addition, the high-speed mechanical operation increases heat generation and easily causes malfunction.

Since fluorescent bodies with different light-emitting bands typically have different excitation wavelength bands, a white light source is used as an excitation light source, a filter on the excitation side is concurrently switched with switching of a detection wavelength band, and a fluorescent body to be detected is efficiently excited in the filter wheel scheme. In such a case, the number of excited wavelength bands is four, which is the same as the number of the detected wavelength bands. Although it is generally difficult to efficiently excite all the four fluorescent bodies in a single wavelength band, it is possible to efficiently excite two fluorescent bodies in a single wavelength band since the excitation wavelength band has a finite range and fluorescent bodies with similar light-emitting wavelengths have overlapped excitation bands. Therefore, it is possible to efficiently excite all the four fluorescent bodies in two wavelength bands.

According to NPL 1, four fluorescent bodies are concurrently excited by two lasers with different wavelengths. The apparatus disclosed in NPL 1 divides light emitted from DNA into four wavelength bands by three dichroic mirrors, and concurrently obtains four images by using four imaging sensors. In addition, since the apparatus disclosed in NPL 1 does not include mechanical filter switching and concurrently captures the four images, the apparatus can obtain images corresponding to one panel in a period of time corresponding to ¼ or less than that in PTL 1 However, usage of the four imaging sensors requires a significantly large-scale and expensive apparatus as compared with the scheme disclosed in PTL 1.

### CITATION LIST

### Patent Literature

PTL 1: US Patent Application No. 2011/0236964

### Non-Patent Literature

NPL 1: Haga T, Sonehara T, Sakai T, Anazawa T, Fujita T, Takahashi S, “Simultaneous four-color imaging of single molecule fluorophores using dichroic mirrors and tour charge-coupled devices”, Rev Sci Instrum. 2011 February; 82 (2):023701. doi:10.1063/1.3524570.

### SUMMARY OF INVENTION

### Technical Problem

An object of the invention is to provide a technique of imaging one panel for a shorter period of time as compared with a filter wheel scheme, without causing significant increases in cost and size.

### Solution to Problem

The present inventors discovered that the aforementioned problem can be solved by alternately illuminating a sample with two light sources having different wavelengths, modifying a nucleic acid in the sample with two types of fluorescent dye that emit light in bandwidths between the wavelengths of the light sources and two types of fluorescent dye that emit light in longer wavelength bands than those of both the light sources, causing the fluorescent light from the nucleic acid to split by a dichroic mirror that has a transition wavelength from transmission to reflection in two locations, namely: between light-emitting bands of two types of short-wavelength fluorescent dye and between light-emitting bands of two types of long-wavelength fluorescent dye and detecting the split light by two detectors.

In order to solve the aforementioned problem, the configurations described in claims, for example, are employed. Although the application includes a plurality of means for solving the problem, one example thereof will be described. There is provided a nucleic-acid-sequence determination device including: two light sources having different wavelengths; two detectors; and an optical system for irradiating a sample with light from the two light sources and guiding fluorescent light from a nucleic acid in the sample to the two detectors, in which the two light sources alternately illuminate the sample and modify the nucleic acid in the sample with two types of fluorescent dye that emit light in bandwidths between the wavelengths of the two light sources and two types of fluorescent dye that emit light in longer wavelength hands than those of the two light sources, and in which the optical system is provided with a dichroic mirror for causing the fluorescent light from the nucleic acid in the sample to split, and guiding the split light to the two detectors, and the dichroic mirror has a transition wavelength from transmission to reflection in two locations, namely: between light-emitting bands of two types of short-wavelength fluorescent dye and between light-emitting bands of two types of long-wavelength fluorescent dye.

According to another example, there is provided a nucleic-acid-sequence determination method including steps of: alternately illuminating a sample with light from two light sources having different wavelengths and modifying a nucleic acid in the sample with two types of fluorescent dye that emit light in bandwidths between the wavelengths of the two light sources and two types of fluorescent dye that emit light in longer wavelength bands than those of the two light sources; causing fluorescent light from the nucleic acid in the sample to split by a dichroic mirror and guiding the split light to two detectors, the dichroic mirror having a transition wavelength from transmission to reflection in two locations, namely: between light-emitting bands of two types of short-wavelength fluorescent dye and between light-emitting bands of two types of long-wavelength fluorescent dye; and capturing at least one sample image by both the two detectors, respectively, while one of the two light sources illuminates the sample.

### Advantageous Effects of Invention

According to the invention, high reliability can be achieved due to no mechanical moving elements, and imaging time becomes half or less to ⅓, as compared with a case of the filter wheel scheme.

Further features in relation to the invention will become obvious from the description in the specification and accompanying drawings. In addition, configurations and effects, as objects, other than those described above will become obvious from the following description of embodiments.

### BRIEF DESCRIPTION OF DRAWINGS

FIG. 1 is an outline configuration diagram of a nucleic-acid-sequence determination device according to a first embodiment of the invention.

FIG. 2 is a schematic diagram of excitation spectra of four types of fluorescent dye and light emitting spectra of light sources.

FIG. 3 is a timing chart illustrating a measurement operation of the nucleic-acid-sequence determination device according to the first embodiment of the invention.

FIG. 4 is a schematic diagram of light-emitting spectra of the four types of fluorescent dye and a transmission spectrum of a dichroic mirror.

FIG. 5 is a timing chart illustrating a measurement operation of a nucleic-acid-sequence determination device according to a second embodiment of the invention.

FIG. 6 is a schematic diagram of light-emitting spectra of four types of fluorescent dye and a transmission spectrum of a dichroic mirror according to a third embodiment of the invention.

### DESCRIPTION OF EMBODIMENTS

Hereinafter, a description will be given of embodiments of the invention with reference to accompanying drawings. Although the accompanying drawings illustrate specific embodiments based on a principle of the invention, the drawings are provided only for understanding of the invention and are not intended to be used for interpreting the invention in a limited manner.

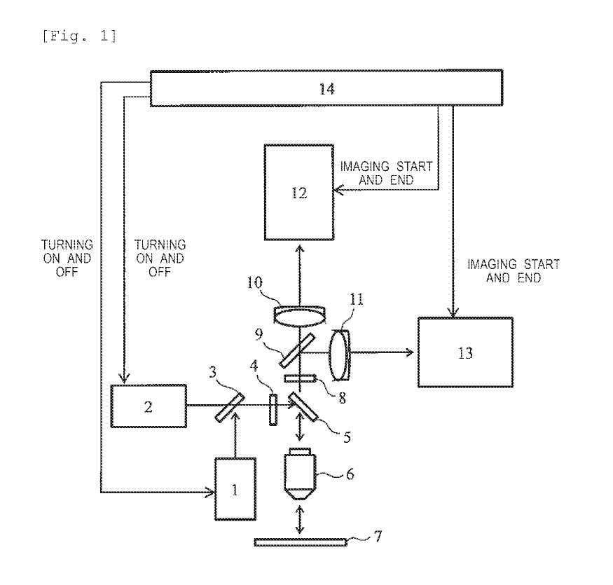

FIG. 1 is an outline configuration diagram of a nucleic-acid-sequence determination device according to a first embodiment. The nucleic-acid-sequence determination device includes first and second semiconductor light sources 1 and 2 that serve as two light sources with different wavelengths, two imaging sensors (detectors) 12 and 13, and an optical system that is for irradiating a sample substrate 7 with light from the first and second semiconductor light sources 1 and 2 and guiding fluorescent light from a nucleic acid in a sample to the two imaging sensors 12 and 13. The optical system includes dichroic mirrors 3, 5, and 9, bandpass filters 4 and 8, an objective lens 6, and first and second camera lenses 10 and 11. In addition, the nucleic-acid-sequence determination device includes a host controller 14 that is for controlling the respective components. Hereinafter, a description will be given of operations of the respective components in the nucleic-acid-sequence determination device.

Light emitted from the first semiconductor light source 1 and the second semiconductor light source 2 is combined by the excitation light combining dichroic mirror 3. For the combined light, skirt parts of light-emitting spectra of the semiconductor light sources inside light-emitting bands of fluorescent dye are blocked by the excitation bandpass filter 4 with two transmitting bands. The bandpass filter 4 transmits light only in the periphery of center wavelengths of the two semiconductor light sources 1 and 2 and blocks transmitting ranges of the bandpass filter 8 which will be described later. This reduces background light and enables high-sensitivity measurement.

According to the embodiment, the first and second semiconductor light sources 1 and 2 are light-emitting diodes (LED) that have center wavelengths of 495 nm and 640 nm, respectively. The first and second semiconductor light sources 1 and 2 respectively have a built-in collimate lens and emit a parallel light flux. As light sources, semiconductor lasers with substantially the same wavelengths may be used. Usage of the light-emitting diodes (LED) or the semiconductor lasers brings about an increase in speed of switching a light source that is turned on.

Although the bandpass filter 4 with the two transmitting bands is installed after the dichroic mirror 3 in the embodiment, it is also possible to provide a bandpass filter with a single transmitting band between the dichroic mirror 3 and the first semiconductor light source 1 and between the dichroic mirror 3 and the second semiconductor light source 2, respectively, to cause the bandpass filters to perform the functions of the bandpass filter 4 instead.

The light transmitted through the bandpass filter 4 is reflected by the dichroic mirror 5 for splitting the light into excitation light and fluorescent light, is introduced into the objective lens 6, and illuminates the sample substrate 7. The sample substrate 7 is arranged on a stage which is not shown in the drawing. A large number of clusters of amplified DNA are formed on the sample substrate 7, and fluorescent dye modifying the DNA is excited and emits fluorescent light.

The fluorescent light emitted from the sample substrate 7 is collected by the objective lens 6, is transmitted through the dichroic mirror 5, and is then transmitted through the fluorescent light bandpass filter 8. The bandpass filter 8 sufficiently blocks the excitation light component. The bandpass filter 8 is configured to block light-emitting wavelengths of the two semiconductor light sources 1 and 2, and transmit a wavelength band between the wavelengths of the two semiconductor light sources 1 and 2 and a longer wavelength band than that of the longer wavelength light source of the two semiconductor light sources 1 and 2. This reduces background light and enables high-sensitivity measurement.

The fluorescent light transmitted through the bandpass filter 8 is divided by the fluorescent dye identifying dichroic mirror 9. A part of the fluorescent light is made to form an image on the first imaging sensor 12 by the first camera lens 10 after being transmitted through the dichroic mirror 9. The other part of the fluorescent light is made to form an image on the second imaging sensor 13 by the second camera lens 11 after being reflected by the dichroic mirror 9. The host controller 14 outputs a control signal for turning on and off the first and second semiconductor light sources 1 and 2 and a control signal for starting and ending the imaging by the imaging sensors 12 and 13, and these control signals are input to the respective targets of control.

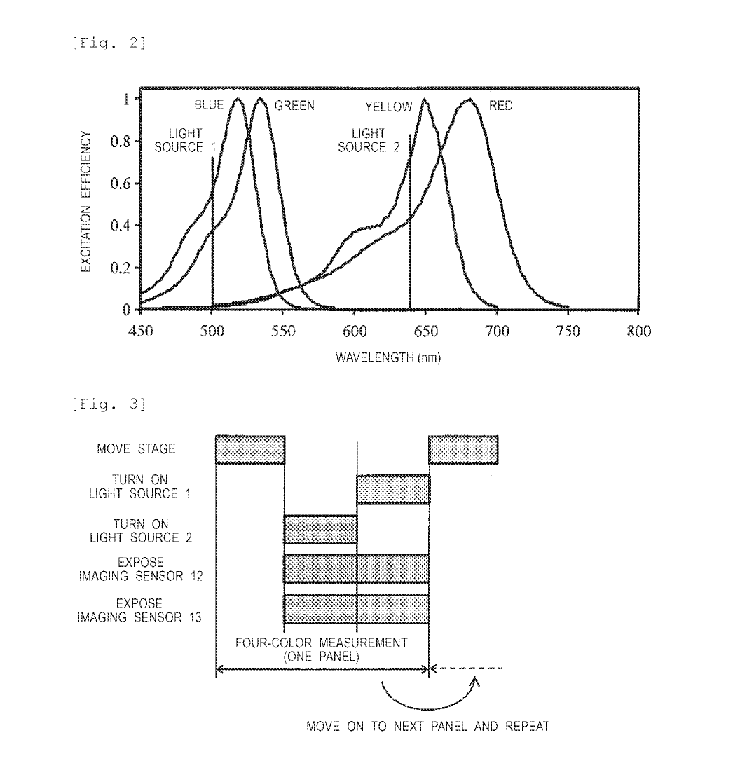

FIG. 2 illustrates excitation spectra of four fluorescent bodies that modify the DNA and wavelength bands output by the first and second semiconductor light sources 1 and 2 according to the embodiment. The first and second semiconductor light sources 1 and 2 alternately irradiate the sample substrate 7. Here, the nucleic acid in the sample is modified by two types of fluorescent dye (blue and green) that emit light in bands between the wavelengths of the two semiconductor light sources 1 and 2 and two types of fluorescent dye (yellow and red) that emit light in longer wavelength bands than those of the two semiconductor light sources 1 and 2. FIG. 2 illustrates that blue and green fluorescent bodies are preferably excited in a wavelength band of 495 nm and yellow and red fluorescent bodies are preferably excited in a wavelength band of 640 nm.

FIG. 3 is a timing chart of imaging one panel according to the embodiment. First, the stage is moved, and the panel to be imaged is moved toward the inside of an observation field of view. Thereafter, only the second semiconductor light source 2 is turned on, and the imaging sensor 12 and the imaging sensor 13 are concurrently exposed for a predetermined period of time. The exposure of the imaging sensors 12 and 13 is completed at the same time as the turning-off of the second semiconductor light source 2, and two image data items (referred to as a first image pair) obtained by the two imaging sensors 12 and 13 are transferred to the host controller 14.

Next, only the first semiconductor light source 1 is turned on, the imaging sensor 12 and the imaging sensor 13 are concurrently exposed for a predetermined period of time again, and the first semiconductor light source 1 is then turned off. Two image data items (referred to as a second image pair) obtained by the two imaging sensors 12 and 13 are transferred to the host controller 14. As described above, at least one sample image data item is captured by each of both the two imaging sensors 12 and 13 while one of the first and second semiconductor light sources 1 and 2 illuminates the sample substrate 7.

Since interline CCDs are used as the imaging sensors 12 and 13 in the embodiment, the exposure and the transfer are performed. In parallel, and the exposure of the second image pair is started at the same time as the end of the exposure of the first image pair. In a case of using image sensors, such as CMOS sensors, other than the interline CCDs, an image data transfer timing is inserted between the first exposure timing and the second exposure timing. However, since recent CMOS sensors perform data transfer at a significantly high speed, it is possible to substantially ignore the image data transfer timing, and to use substantially the same timing chart as that in FIG. 3.

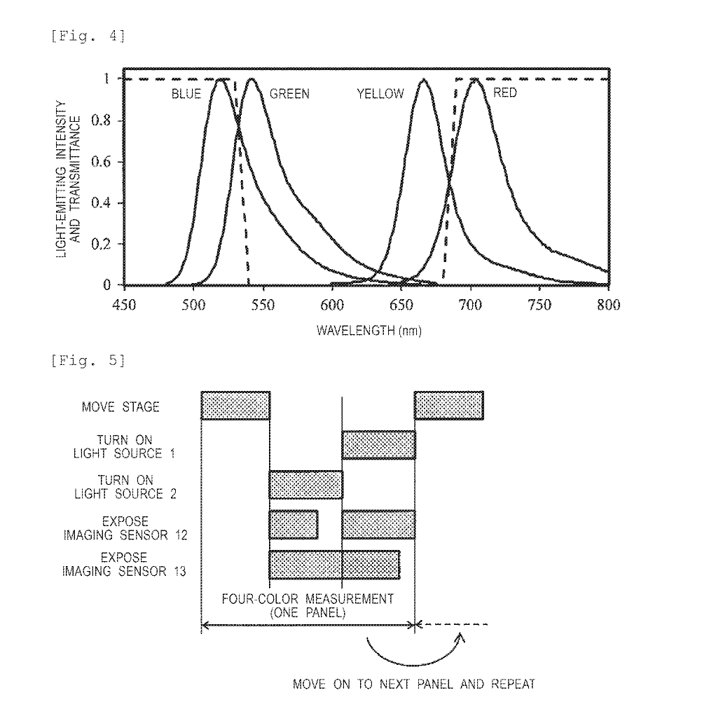

FIG. 4 illustrates light-emitting spectra of the four fluorescent bodies and a transmitting spectrum of the dichroic mirror 9. Absorption loss at the dichroic mirror 9 can be ignored, and a reflection rate can be regarded as (1-transmittance). The dichroic mirror 9 has a transition wavelength from transmittance to reflectance in two locations, namely between light-emitting bands of the two types of short-wavelength fluorescent dye (blue and green) and between light-emitting bands of the two types of long-wavelength fluorescent dye (yellow and red). As illustrated in FIG. 2, only the yellow and red fluorescent bodies are substantially excited and emit light when only the second semiconductor light source 2 is turned on. As illustrated in FIG. 4, most of the light emitted by the yellow fluorescent body is reflected by the dichroic mirror 9 and is detected by the imaging sensor 13, and most of the light emitted by the red fluorescent body is transmitted through the dichroic mirror 9 and is detected by the imaging sensor 12.

In contrast, only the blue and green fluorescent bodies are substantially excited and emit light when only the first semiconductor light source us turned on. As illustrated in FIG. 4, most of the light emitted by the blue fluorescent body is transmitted through the dichroic mirror 9 and is detected by the imaging sensor 12, and most of the light emitted by the green fluorescent body is reflected by the dichroic mirror 9 and is detected by the imaging sensor 13.

It is assumed that a value obtained by subtracting luminance of a background from luminance of a luminescent spot in an image corresponding to each cluster is referred to as a signal. Four-dimensional vectors (a signal obtained by the imaging sensor 12 when the first semiconductor light source 1 is turned on, a signal obtained by the imaging sensor 13 when the first semiconductor light source 1 is turned on, a signal obtained by the imaging sensor 12 when the second semiconductor light source 2 is turned on, and a signal obtained by the imaging sensor 13 when the second semiconductor light source 2 is turned on) are referred to as signal vectors. The signal vectors corresponding to the respective fluorescent bodies obtained in the embodiment are as follows.

Blue signal vector=(0.59, 1.31, 0.00, 0.00)

Green signal vector=(0.05, 0.44, 0.00, 0.00)

Yellow signal vector=(0.00, 0.00, 0.63, 0.35)

Red signal vector=(0.00, 0.00, 0.16, 0.07)

The blue and green light-emitting spectra overlap each other, and the yellow and red light-emitting spectra overlap each other. As a result, the four signal vectors do not completely orthogonally intersect but the signal vectors are sufficiently independent. Therefore, it is possible to determine types of fluorescent bodies with high precision based on the signal vectors of the cluster.

In the related art, there are four exposure timings if a four-color image is captured based on a general filter wheel scheme, and it takes a long time due to time for switching filters. In addition, there is also a problem that a speed of mechanical switching of the filters increases and an increase in heat generation occurs in response to a requirement for an increase in the speed to the maximum extent. According to the embodiment, it is possible to provide a nucleic-acid-sequence determination device that can achieve high reliability due to no mechanical moving elements and reduce imaging time to half or less to ⅓ merely by causing slight increases in cost and size as compared with the filter wheel scheme.

Since there are two exposure timings for one panel in the embodiment, for example, the time required for one panel increases as compared with that in NPL 1 However, the time is not doubled and is about 1.5 times as long as that in NPL 1 if an operation time of the stage is taken into consideration. In contrast, since the number of imaging sensors is reduced from our to two, the cost of the device becomes about half, and the size of the device is significantly reduced. Although one extra imaging sensor is required as compared with the filter wheel scheme, this causes a slight increase in the cost, and the time required for one panel becomes less than half since a filter wheel rotation mechanism is not provided. According to the embodiment, it possible to greatly enhance performance by causing a slight increase in the cost as compared with the filter wheel scheme and to construct a nucleic-acid-sequence determination device that can achieve excellent cost performance and high reliability due to no high-speed rotation mechanism as described above.

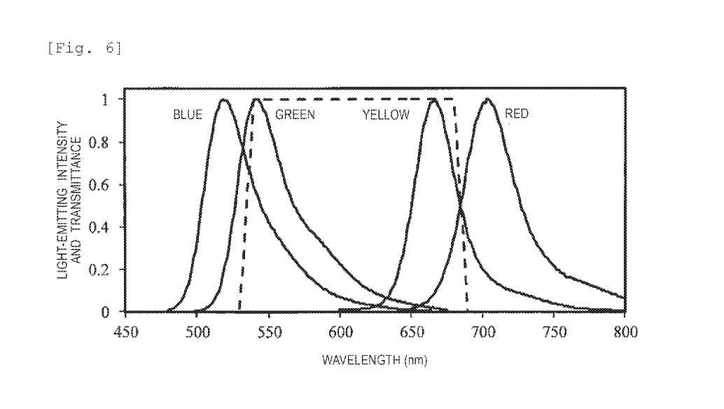

FIG. 5 is a timing chart of imaging one panel according to a second embodiment of the invention. A configuration of a nucleic-acid-sequence determination device according to the embodiment is the same as that in the first embodiment.

There is a case in which if two fluorescent bodies are excited in one wavelength band, light-emitting intensities are significantly different from each other due to different excitation efficiency. In a case of exciting the fluorescent bodies one by one in a time-division manner or with two independent light sources, it is possible to obtain the same intensity by adjusting intensity of the light sources or adjusting transmittance of the excitation filter for the respective fluorescent bodies. However, the scheme of the invention cannot employ such a method. Therefore, in a case in which light-emitting intensities of two concurrently excited fluorescent bodies are significantly different from each other and the exposure time of the two imaging devices is the same, S/N of one fluorescent body becomes lower (if it is attempted to raise SN of the fluorescent body with lower light-emitting intensity, then the signal from the fluorescent body with higher light-emitting intensity is saturated).

According to the embodiment, the exposure times of the two imaging sensors 12 and 13 while one of the two semiconductor light sources 1 and 2 illuminates the sample are different from each other. Setting the different exposure time for the two imaging sensors 12 and 13 has an advantage that both the images can be obtained with satisfactory S/N even in a case in which light-emitting intensities of the fluorescent bodies greatly differ from each other. As illustrated in FIG. 5, the exposure of the two imaging sensors 12 and 13 is concurrently started, and the exposure of the imaging sensor 12, for which the exposure time is set to be shorter, is completed first in the embodiment. However, the invention is not limited thereto, and the same effect can completely be achieved even by delaying the start of the exposure of the imaging sensor 12, for which the exposure time is set to be shorter

FIG. 6 illustrates light-emitting spectra of four fluorescent bodies and a transmission spectrum of the dichroic mirror 9 according to a third embodiment. A configuration of a nucleic-acid-sequence determination device and fluorescent bodies in the embodiment are the same as those in the first embodiment. In relation to the transmission spectrum of the dichroic mirror 9, transmission and reflection are reversed from those in the first embodiment. It is possible to separately detect the fluorescent light from the two fluorescent bodies that emit light concurrently in light substantially the same manner as those in the first embodiment, even in the case of such a transmission property.

Substantially only the yellow and red fluorescent bodies are excited and emit light when only the second semiconductor light source 2 is turned on. As illustrated in FIG. 6, most of the light emitted by the yellow fluorescent body is transmitted through the dichroic mirror 9 and is detected by the imaging sensor 12, and most of the light emitted by the red fluorescent body is reflected by the dichroic mirror 9 and is detected by the imaging sensor 13. In contrast, substantially only the blue and green fluorescent bodies are excited and emit light when only the first semiconductor light source 1 is turned on. As illustrated in FIG. 6, most of the light emitted by the blue fluorescent body is reflected by the dichroic mirror 9 and is detected by the imaging sensor 13, and most of the light emitted by the green fluorescent body is transmitted through the dichroic mirror 9 and is detected by the imaging sensor 12.

According to the invention, it is possible to provide a nucleic-acid-sequence determination device that can achieve high reliability due to no mechanical moving elements and reduce imaging time to half or less to ⅓ merely by causing slight increases in cost and size as compared with the filter wheel scheme.

The invention is not limited to the aforementioned embodiments, and various modifications are included. For example, the aforementioned embodiments were described in detail for clearly explaining the invention, and the invention is not necessarilly limited to a structure provided with all the aforementioned configurations. In addition, a part of a configuration of an embodiment may be replaced with a configuration of another embodiment, and a configuration of another embodiment can be added to a configuration of an embodiment. Moreover, addition, deletion, and replacement of another configuration can be made on a part of a configuration in each embodiment.

A part or the entirety of the respective configurations, functions, processing units, and the like of the host controller 14 may be realized as hardware by designing the part or the entirety as an integrated circuit, for example. In addition, the respective configurations, functions, processing units, and the like of the host controller 14 may be realized as software by causing a processor to interpret and execute a program that realizes the respective functions. Information including programs, tables, and files for realizing the respective functions can be stored in a storage device such as a memory, a hard disk, or a solid, state drive (SSD) or a recording medium such as an IC card, an SD card, or a DVD.

Control lines and information lines that were necessary for explanation were described in the aforementioned embodiments, and all the control lines and the information lines of the product were not necessarily described. It may be considered that substantially all the configurations are connected to each other in practice.

### REFERENCE SIGNS LIST

1: first semiconductor light source
2: second semiconductor light source
3: excitation light combining dichroic mirror
4: excitation light bandpass filter
5: excitation light/fluorescent light separating dichroic mirror
6: objective lens
7: sample substrate
8: fluorescent light bandpass filter
9: fluorescent dye identifying dichroic mirror
10: first camera lens
11: second camera lens
12: first imaging sensor
13: second imaging sensor
14: host controller

1. A nucleic-acid-sequence determination device comprising:
two light sources having different wavelengths;
two detectors; and
an optical system for irradiating a sample with light from the two light sources and guiding fluorescent light from a nucleic acid in the sample to the two detectors,
wherein the two light sources alternately illuminate the sample and modify the nucleic acid in the sample with two types of fluorescent dye that emit light in bandwidths between the wavelengths of the two light sources and two types of fluorescent dye that emit light in longer wavelength bands than those of the two light sources, and
wherein the optical system is provided with a dichroic mirror for causing the fluorescent light from the nucleic acid in the sample to split, and guiding the split light to the two detectors, and the dichroic mirror has a transition wavelength from transmission to reflection in two locations, namely: between light-emitting bands of two types of short-wavelength fluorescent dye and between light-emitting bands of two types of long-wavelength fluorescent dye.

2. The nucleic-acid-sequence determination device according to claim 1,
wherein the optical system is provided with a first filter that blocks light-emitting wavelengths of the two light sources and transmits a wavelength band between the wavelengths of the two light sources and a longer wavelength band than that of the longer wavelength light source of the two light sources.

3. The nucleic-acid-sequence determination device according to claim 2,
wherein the optical system is provided with a second filter that transmits only a periphery of a center wavelength of the two light sources and blocks transmitted bands of the first filters.

4. The nucleic-acid-sequence determination device according to claim 1,
wherein both the two detectors capture at least one sample image, respectively, while one of the two light sources illuminates the sample.

5. The nucleic-acid-sequence determination device according to claim 1,
wherein the two light sources are light-emitting diodes or semiconductor lasers.

6. The nucleic-acid-sequence determination device according to claim 1, further comprising:
a control device that controls turning-on of the two light sources and sample image capturing by the two detectors.

7. A nucleic-acid-sequence determination method comprising steps of:
alternately illuminating a sample with light from two light sources having different wavelengths and modifying a nucleic acid in the sample with two types of fluorescent dye that emit light in bandwidths between the wavelengths of the two light sources and two types of fluorescent dye that emit light in longer wavelength bands than those of the two light sources;
causing fluorescent light from the nucleic acid in the sample to split by a dichroic mirror and guiding the split light to two detectors, the dichroic mirror having a transition wavelength from transmission to reflection in two locations, namely: between light-emitting bands of two types of short-wavelength fluorescent dye and between light-emitting bands of two types of long-wavelength fluorescent dye; and
capturing at least one sample image by both the two detectors, respectively, while one of the two light sources illuminates the sample.

8. The nucleic-acid-sequence determination method according to claim 7,
wherein in the step of capturing, exposure times of the two detectors while one of the two light sources illuminates the sample are different from each other.
